(19) United States
(12) Patent Application Publication
(10) Pub. No.: US 2016/0228586 A1
(43) Pub. Date: Aug. 11, 2016

(54) Compounds and Their Use for Preparation of Tau Imaging Agents and Tau Imaging Formulations

(71) Applicant: ELI LILLY AND COMPANY, Indianapolis, IN (US)

(72) Inventors: Giorgio ATTARDO, Philadelphia, PA (US); John LISTER-JAMES, Phoenixville, PA (US); Nathaniel Anthony Co LIM, Springfield, PA (US); Hui XIONG, Chesterbrook, PA (US)

(21) Appl. No.: 15/022,657

(22) PCT Filed: Sep. 19, 2014
(86) PCT No.: PCT/US14/56503

§ 371 Date: Mar. 17, 2016

Related U.S. Application Data
(60) Provisional application No. 61/882,769, filed on Sep. 26, 2013.

(51) Int. Cl. A61K 51/04; C07B 59/00; C07C 309/04; C07C 309/30; C07D 471/04

(52) U.S. Cl. CPC A61K 51/0455; C07C 309/30; C07D 471/04; C07C 309/04; C07B 59/002; C07B 2200/05

(57) ABSTRACT

The present invention provides novel trimethylammonium compounds, of the Formula: (I) methods of making these compounds, methods of using the compounds for preparation of tau imaging agents, and preparation of tau imaging agent formulations.

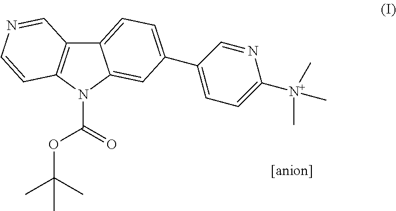 [SMILES: CC(C)(C)OC(=O)n1c2ccncc2c2ccc(-c3ccc([N+](C)(C)C)nc3)cc21]

The present invention relates to novel trimethylammonium compounds, to methods of using the compounds for preparation of the tau imaging agent [¹⁸F]T807, and to compositions and formulations of those preparations for diagnostic imaging, and to methods of imaging using those compounds, compositions, and formulations.

Alzheimer's disease (AD), a leading cause of dementia, develops in one percent of the population between the ages 65 and 69, and increasing to 40-50% in those 95 years and older. AD patients exhibit telltale clinical symptoms that include cognitive impairment and deficits in memory function. In these patients, the presence of AD is confirmed by heavy senile plaque burden found in the cerebral cortex upon post mortem histopathological examination. The mature senile plaques consist of intracellular neurofibrillary tangles (NFT) derived from filaments of hyperphosphorylated tau proteins, and extracellular β-amyloid peptides derived from enzymatic processing of amyloid precursor protein. Aggregates of hyperphosphorylated tau (PHF-tau), such as neurofibrillary tangles, are linked to the degree of cognitive impairment in Alzheimer's disease. [¹⁸F]T807 is a PET imaging agent with demonstrated high affinity and selectivity to PHF-tau, as well as favorable in vivo properties. ([(18)F]T807, a novel tau positron emission tomography imaging agent for Alzheimer's disease. Alzheimer's & Dementia (February 2013) 1-11, available online at http://dx.doi.org/10.1016/j.jalz.2012.11.008).

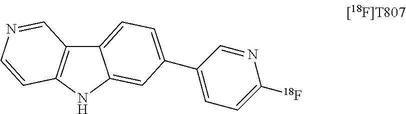 [SMILES: [18F]c1ccc(-c2ccc3c(c2)[nH]c2ccncc23)cn1]

[¹⁸F]T807 is useful for detecting and/or quantitation of tau deposits in patients (Early clinical PET imaging results with the novel PHF-tau radioligand [F-18]-T807, Chien et al., J Alzheimers Dis. 2013 Jan. 1; 34(2):457-68).

There are several potential benefits of imaging tau in the brain with [¹⁸F]T807. Tau imaging will improve diagnosis by identifying potential patients, those having high levels of tau in the brain, who may have increased chance of developing AD. Imaging with [¹⁸F]T807 will also be useful to monitor tau accumulation, and or progression of AD, and when anti-tau drug treatments become available, tau imaging may provide an essential tool for monitoring treatment. Tangles containing tau first appear in brain regions that are very closely linked to memory, and pathologic studies show that tangles may correlate even more strongly with cognition than plaques. Thus, simple noninvasive methods, for detecting and/or quantitation of tau deposits in patients are eagerly sought. (See M. Maruyama et al., “Imaging of tau pathology in a tauopathy mouse model and in Alzheimer patients compared to normal controls”, Neuron, 79: 1094-1108, 2013, C. Mathis and W. Klunk, “Imaging Tau Deposits In Vivo: Progress in Viewing More of The Proteophaty Picture”, Neuron, 79: 1035-10-37, 2013).

Improved technology advancing the capacity to image tau in patients is thus also needed to expand the clinical benefits and impact of diagnostic tau imaging agents. Methods for [¹⁸F]T807 radiosynthesis are known in the art. Shoup et al. recites a method wherein a precursor compound, either an unprotected or a tertbutyl 7-(6-nitropyridin-3-yl)-5H-pyrido[4,3-b]indole-5-carboxylate is radiolabeled via reaction with ¹⁸F with an isocratic HPLC purification (J. Label Compd. Radiopharm (2013)).

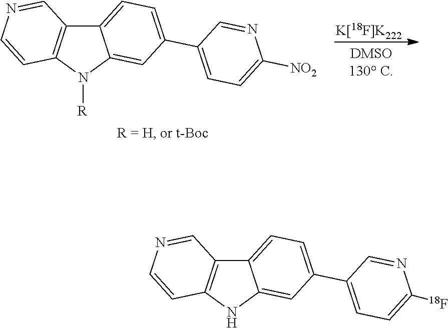

The t-boc version of this precursor, namely tertbutyl 7-(6-nitropyridin-3-yl)-5H-pyrido[4,3-b]indole-5-carboxylate, is shown below.

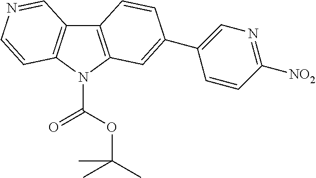 [SMILES: CC(C)(C)OC(=O)n1c2ccncc2c2ccc(-c3ccc([N+](=O)[O-])nc3)cc21]

Xia et al. recites a method wherein the precursor compound is 7-(6-nitropyridin-3-yl)-5H-pyrido[4,3-b]indole, shown below.

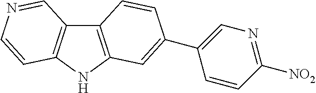 [SMILES: O=[N+]([O-])c1ccc(-c2ccc3c(c2)[nH]c2ccncc23)cn1]

Xia et al. recites that 7-(6-nitropyridin-3-yl)-5H-pyrido[4,3-b]indole is radiolabeled with ¹⁸F using a second step with iron powder/formic acid, in a separate vial, to reduce the nitro group on the remaining precursor to the respective 2-amino-pyridine derivative, thus facilitating separation by HPLC ([(18)F]T807, a novel tau positron emission tomography imaging agent for Alzheimer's disease. Alzheimer's & Dementia (February 2013) 1-11, available online at http://dx.doi.org/10.1016/j.jalz.2012.11.008).

While these methods provide means to prepare [¹⁸F]T807, they have technical attributes that could be improved by the design of innovative synthetic reagents and processes for synthesis of [¹⁸F]T807 to be used in clinical imaging. Improved agents, processes for preparation of [¹⁸F]T807, and imaging formulations, with desirable radiochemistry and/or radiopharmaceutical properties, would be useful for clinical tau imaging. This technology would enhance detection, diagnosis, monitoring and/or management of AD, and other tauopathies. Improved precursor compounds for synthesis of [¹⁸F]T807, having advantageous radiosynthetic properties, would provide enhanced access to [¹⁸F]T807, while avoiding the difficulties associated with existing precursors, and would thus create improved means to produce [¹⁸F]T807, and improved formulations thereof.

The present invention provides the use of compounds of formula I, Ia, or Ib, for the manufacture of a radiopharmaceutical agent [¹⁸F]T807 for imaging of tau in humans. In another aspect the invention provides methods of preparing compounds of formula I, Ia, or Ib. In another aspect the invention provides methods of preparing [¹⁸F]T807 from compounds of formula I, Ia, or Ib. Particularly preferred is the method of preparing [¹⁸F]T807 from the compound of formula Ia. In another aspect the invention provides a pharmaceutical composition comprising [¹⁸F]T807 prepared from a compound of formula I, Ia, or Ib, and a pharmaceutically acceptable diluent or carrier. In another aspect the invention provides a pharmaceutical composition comprising [¹⁸F]T807 prepared from a compound of formula I, Ia, or Ib, which is formulated in 10% (v/v) ethanol/90% w/v (0.9% aqueous Sodium Chloride), preferably for use in humans. The present invention also provides methods of imaging tau comprising introducing into a patient a detectable quantity of [¹⁸F]T807 prepared from a compound of formula I, Ia, or Ib, or a composition thereof.

The present invention provides a compound of formula I:

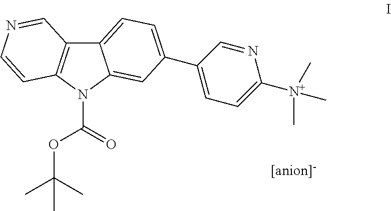 [SMILES: CC(C)(C)OC(=O)n1c2ccncc2c2ccc(-c3ccc([N+](C)(C)C)nc3)cc21]

wherein [anion]⁻ is a suitable anionic counterion. Suitable anionic counterions include non-nucleophilic anions such as organic sulfonates or tartrate. The organic sulfate is preferably an alkyl sulfonate or aryl sulfonate.

The present invention further provides a compound of formula I wherein [anion]⁻ is an alkyl sulfonate or aryl sulfonate. Alkyl sulfonates of the present invention include C₁-C₄ alkyl sulfonate. Aryl sulfonates of the present invention include phenyl sulfonate, wherein the phenyl group is optionally substituted once with C₁-C₄ alkyl, halogen or nitro. Particular values of C₁-C₄ alkyl sulfonate include methanesulfonate (mesylate) and ethanesulfonate. Particular values of phenyl sulfonate include benzenesulfonate, 4-methylbenzenesulfonate (tosylate), 4-bromobenzenesulfonate and 4-nitrobenzenesulfonate. Another suitable anionic counterion is trifluoromethylsulfonate (CF₃SO₃⁻).

A preferred species of the present invention is a compound of formula Ia wherein [anion]⁻ is 4-methylbenzenesulfonate.

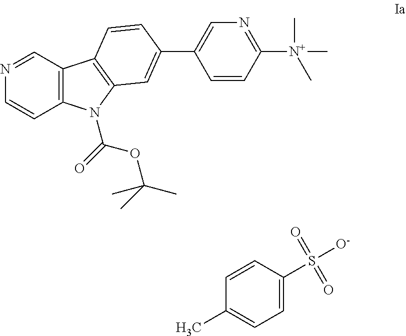

A preferred species of the present invention is a compound of formula Ib wherein [anion]⁻ is methanesulfonate.

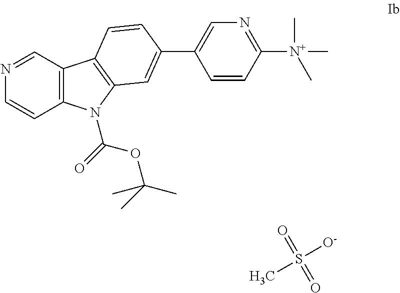

The compounds of formulae I, Ia and Ib are useful, for example, to synthesize a compound of formula II.

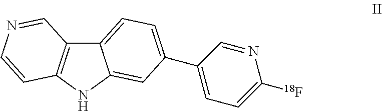 [SMILES: [18F]c1ccc(-c2ccc3c(c2)[nH]c2ccncc23)cn1]

The compound of formula II is also referred to as [¹⁸F]T807.

The present invention provides a compound of formula II prepared from a compound of formula I:

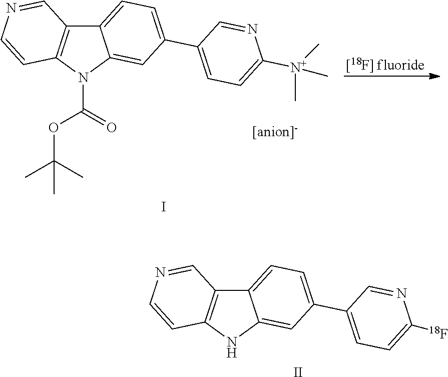

The invention further provides a compound of formula II prepared from a compound of formula Ia or formula Ib.

The present invention provides a process of making a compound of the formula:

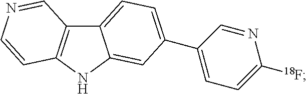

comprising reacting 5-(5-(tert-butoxycarbonyl)-5H-pyrido[4,3-b]indol-7-yl)-N,N,N-trimethylpyridin-2-aminium 4-methylbenzenesulfonate, represented by the formula:

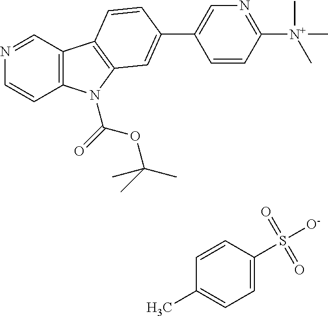

with a source of [¹⁸F]fluoride.

The following Schemes, Preparations, and Examples are provided to better elucidate the practice of the present invention. Suitable reaction conditions for the steps of these Schemes, Preparations, and Examples are well known in the art and appropriate modification of reaction conditions, including substitution of solvents and co-reagents are within the ability of the skilled artisan.

### General Chemistry

A compound of formula II may be prepared from a compound of formula I. More specifically as shown in Scheme 1, a compound of formula Ia is first reacted with a suitable source of [¹⁸F]fluoride such as Cryptand 2.2.2-K₂CO₃ [¹⁸F]fluoride in the presence of a base such as potassium carbonate. The reaction is conveniently carried out in a solvent such as DMSO, acetonitrile, and mixtures thereof. The resulting N-protected [¹⁸F] intermediate is reacted with a suitable acid such as aqueous hydrochloric acid in a solvent such as DMSO and water to provide a compound of formula II.

A compound of formula I may be prepared from a compound of formula III. More specifically, a compound III is reacted with an activator such as p-toluenesulfonic anhydride (tosic anhydride) or trifluoroacetic anhydride and trimethyl amine in a solvent such as methylene chloride to provide a compound of formula Ia where [anion]⁻ is 4-methylbenzenesulfonate, or alternatively trifluoroacetate. The use of pyridine, triazole, trialkyl or heteroaryl amines, results in the pyridinium, triazolium, trialkylammonium, or heterocyclic ammonium compounds respectively. A compound of formula III may be prepared from a compound of formula IV. More specifically, a compound of formula IV is reacted with bis(pinacolato)diboron in the presence of a transition metal catalyst such as dichloro 1,1′-bis(diphenylphosphino)ferrocene palladium (II) dichloromethane adduct in dioxane. The resulting pinacol ester intermediate is reacted with 3-bromo-pyridine 1-oxide in the presence of a catalyst such as palladium(II) tetrakistriphenylphosphine and a base such as aqueous sodium carbonate to provide a compound of formula III. The reaction is conveniently carried out in a solvent such as methylene chloride.

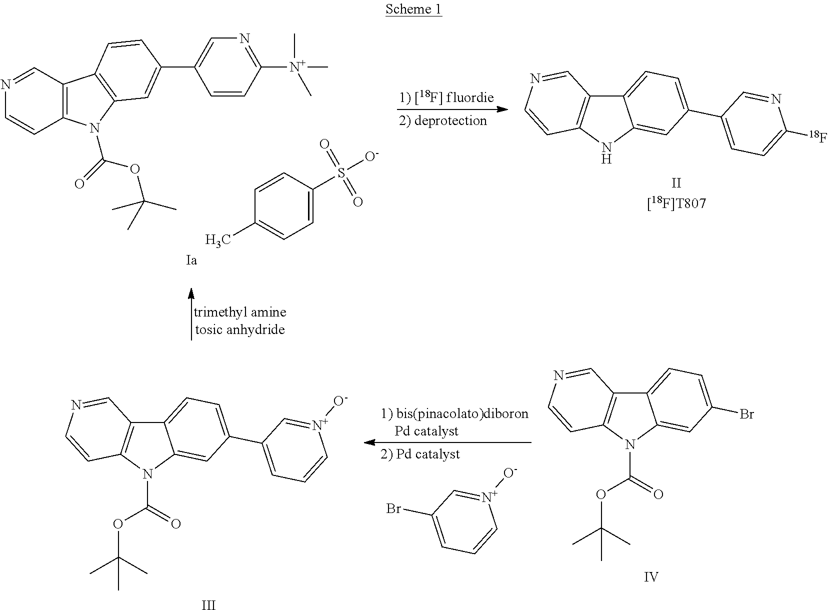

Furthermore, the skilled artisan will appreciate that in some circumstances, the order in which moieties are introduced is not critical. The particular order of steps required to produce the compounds of Formula I is dependent upon the particular compound being synthesized, the starting compound, and the relative lability of the substituted moieties, as is well appreciated by the skilled chemist. The skilled artisan will appreciate that not all substituents are compatible with all reaction conditions. These compounds may be protected or modified at a convenient point in the synthesis by methods well known in the art. The intermediates and final products of the present invention may be further purified, if desired by common techniques such as recrystallization or chromatography over solid supports such as silica gel or alumina.

The compounds of the present invention are preferably formulated as radiopharmaceutical compositions administered by a variety of routes. Preferably, such compositions are for intravenous use. Such pharmaceutical compositions and processes for preparing same are well known in the art. See, e.g., Remington: The Science and Practice of Pharmacy (A. Gennaro, et al., eds., 19^{th} ed., Mack Publishing Co., 1995).

Preferred formulations of the present invention are preparations of [¹⁸F]T807 prepared from compounds of formula I, and particularly preferred are formulations of [¹⁸F]T807 prepared from the compound of formula Ia. Particularly preferred is [¹⁸F]T807 prepared from the compound of formula Ia according to the procedures described herein according to Scheme 1. Particularly preferred is [¹⁸F]T807 prepared from the compound of formula Ia according to the procedures described herein according to Example 1 and Example 2. A preferred formulation of [¹⁸F]T807 is prepared from a compound of formula I and formulated in 10% (v/v) ethanol/90% w/v (0.9% aqueous Sodium Chloride). A particularly preferred formulation of [¹⁸F]T807 is prepared from the compound of formula Ia and formulated in 10% (v/v) ethanol/90% w/v (0.9% aqueous Sodium Chloride). A preferred formulation of [¹⁸F]T807 is prepared from a compound of formula I and formulated in 10% (v/v) ethanol/90% (21 mM sodium phosphate). A preferred formulation of [¹⁸F]T807 is prepared from a compound of formula Ia and formulated in 10% (v/v) ethanol/90% (21 mM sodium phosphate). Another embodiment of the invention is a formulation of [¹⁸F]T807 prepared from the compound of formula Ia and formulated in 9% (v/v) ethanol, 1% (w/v) Kolliphor HS 15, and 90% (v/v) (0.9% aqueous Sodium Chloride). Particularly preferred is [¹⁸F]T807 prepared from the compound of formula Ia according to the procedures described herein according to Scheme 1 and formulated in 10% (v/v) ethanol/90% w/v (0.9% aqueous Sodium Chloride). Particularly preferred is [¹⁸F]T807 prepared from the compound of formula Ia according to the procedures described herein according to Example 1 and Example 2 and formulated in 10% (v/v) ethanol/90% w/v (0.9% aqueous Sodium Chloride).

Novel trimethylammonium compounds of the present invention have been discovered to be surprisingly and unexpectedly advantageous for use as synthetic precursors for the radiosynthesis of [¹⁸F]T807 for imaging uses, including human clinical imaging. A preferred compound, the compound of formula Ia, possesses a combination of particularly useful properties as a precursor for synthesis of [¹⁸F]T807, including solubility, reactivity, shorter reaction times, separability, and yield. This surprisingly advantageous combination of improved properties leads to an effective and efficient clinical process for radiosynthesis of [¹⁸F]T807 which facilitates imaging of patients for tau burden. The solubility of the precursor compound affects the ability of the compound to get into solution so that a reaction to produce [¹⁸F]T807 can effectively occur. In contrast to 7-(6-nitropyridin-3-yl)-5H-pyrido[4,3-b]indole, the compound of formula Ia is readily soluble in DMSO, and therefore does not require sonication or heating to get the compound into solution, as is required for prior methods using the precursor 7-(6-nitropyridin-3-yl)-5H-pyrido[4,3-b]indole.

Synthesis of [¹⁸F]T807 from 7-(6-nitropyridin-3-yl)-5H-pyrido[4,3-b]indole by the non-iron method also has a drawback in that leftover un-reacted 7-(6-nitropyridin-3-yl)-5H-pyrido[4,3-b]indole precipitates during aqueous reaction workup. This precipitation can lead to blocks in the fluidic pathway and can ultimately result in production failure. In contrast, the improved solubility of the compound of formula Ia reduces and/or eliminates the risk of production failures due to precipitation. Production failures are a known problem in the clinical practice of radiosynthesis and can limit the number of batches that can be prepared on a daily basis, and thus limit the number of patients that can be imaged in a window of time. Production failures thus can have important impact on the cost, accessibility, and convenience of patient imaging.

Yield is another important aspect of a clinical radiosynthetic process for preparation of [¹⁸F]T807. The process using the compound of formula Ia results in clinically useful yields. In contrast, bromo or chloro substituted precursors have very low corrected yields (<5%), as the bromo and chloro substituents are not readily displaceable under normal, no carrier added, nucleophilic aromatic fluorination conditions. While this aspect alone is significant and important, this property, when discovered to be in combination with other advantageous properties of the compound of formula Ia, results in surprising improvements in clinical radiosynthetic processes for preparation of [¹⁸F]T807.

Further, product yields can be negatively impacted by the difficulty to destroy the left over un-reacted 7-(6-nitropyridin-3-yl)-5H-pyrido[4,3-b]indole. For instance, the iron-process using 7-(6-nitropyridin-3-yl)-5H-pyrido[4,3-b]indole to produce [¹⁸F]T807 at two different production sites, corrected yields are obtained of 42%, 48% and 25% at Culver City, and 8%, 6% and 19% at Northwales. In comparison, the Siemens non-iron process results in corrected yields of 71%, 45%, 70%, 55% and 54%. These results indicate during destruction of 7-(6-nitropyridin-3-yl)-5H-pyrido[4,3-b]indole, an accompanying destruction of [¹⁸F]T807 can occur which leads to yield inconsistencies and lower corrected yields. In contrast, using the compound of formula Ia, only requires removal of the boc-protecting group under milder chemical conditions, and the separation of the non-boc protected positively charged compound of formula Ia from [¹⁸F]T807, which leads to consistently high corrected product yields of 45-55%.

The ability to effectively and efficiently purify the [¹⁸F]T807 product is the third important attribute of a clinical radiosynthetic process for preparation of [¹⁸F]T807, and this aspect can be impacted by the precursor used in the process. A process for preparation of [¹⁸F]T807 using 7-(6-nitropyridin-3-yl)-5H-pyrido[4,3-b]indole requires destruction of leftover un-reacted 7-(6-nitropyridin-3-yl)-5H-pyrido[4,3-b]indole in order to facilitate chromatographic purification of the product [¹⁸F]T807. This is because 7-(6-nitropyridin-3-yl)-5H-pyrido[4,3-b]indole and [18F]T807 have similar chromatography properties making it challenging to separate one from the other. In the Siemens non-iron process using 7-(6-nitropyridin-3-yl)-5H-pyrido[4,3-b]indole, where high levels of 7-(6-nitropyridin-3-yl)-5H-pyrido[4,3-b]indole remain after the reaction, the HPLC mobile phase composition is restricted to contain less organic solvent in order to allow separation. This relative lack of organic solvent leads to an [¹⁸F]T807 elution from the separation column at a time around 27 minutes. This relatively long time is disadvantageous for short-lived radionuclide labeled radiopharmaceuticals such as [¹⁸F]T807. In contrast, the present process using the compound of formula Ia allows for [¹⁸F]T807 to be eluted at 8 minutes with little to no co-eluting impurities. Further, the significant differences in chromatography properties between [¹⁸F]T807 and by-product generated using the compound of formula Ia precursor also facilitate the use of cartridge-based purification as opposed to more tedious and time consuming HPLC processes. Thus, use of the compound of formula Ia can also simplify the production process. These differences can result in significantly faster overall production times, and increases clinical radiosynthesis production capacity, which can have important positive impact on the cost, accessibility, and convenience of patient imaging.

Thus, the present process using the compound of formula Ia, 5-(5-(tert-butoxycarbonyl)-5H-pyrido[4,3-b]indol-7-yl)-N,N,N-trimethylpyridin-2-aminium 4-methylbenzenesulfonate, has unexpected and important real world advantages for the reliability and production capacity in clinical radiosynthesis of [¹⁸F]T807 imaging doses. More batches, from improved capacity and reliability, can have important positive impact on the ability to image tau in patients.

### DESCRIPTION OF THE FIGURES

FIG. 1: HPLC peak matching profile of 7-[¹⁸F]fluoro-5H-pyrido[4,3-b]indole, [¹⁸F]T807. Upper panel labelled [¹⁸F]T807 (HPLC Gamma Detector) illustrates the radio-chromatogram of 7-(6-[¹⁸F]fluoro-3-pyridyl)-5H-pyrido[4,3-b]indole, [¹⁸F]T807. Lower panel labelled T807 (HPLC UV Detector) illustrates the ultraviolet chromatogram of 7-(6-[¹⁸F]fluoro-3-pyridyl)-5H-pyrido[4,3-b]indole, [¹⁸F]T807.

FIG. 2: [¹⁸F]T807 labeling of tau in post-mortem frontal lobe sections (10 um) of Tau positive human brain tissue from patients with AD, approximately 20 uCi of [¹⁸F]T807 per slide. Strong autoradiography signal of [¹⁸F]T807 is observed on the grey matter (GM) region, and the presence of tau in these regions are confirmed by tau-immunostaining. The non-specific or background [¹⁸F]T807 signal is shown in the white matter region (WM) and is low. The specificity of the autoradiography signal, with respect to binding to native tau aggregates, is indicated by the blocking effect of 1 uM of cold T807.

### EXAMPLES AND PREPARATIONS

All reactions are run under a nitrogen atmosphere unless otherwise noted. Products are purified using an automated Teledyne Isco® Flash Chromatography System. Reagents, solvents, and supplies are known to the skilled chemist.

¹H and ¹³C NMR spectra are recorded on a Bruker HD Avance III 400 spectrometer in CDCl₃ (Cambridge Isotope Laboratories, Cat. No. DLM-7-100, passed over basic alumina just prior to use) or DMSO-d₆, (Cambridge Isotope Laboratories, Cat. No. DLM-10-25). HRMS data is obtained on a Waters QT of mass spectrometer using an electrospray ionization positive scan mode. Elemental analysis is performed at Galbraith Laboratories (GLI) (Galbraith Inc., 2323 Sycamore Drive, Knoxville, Tenn. 37921) using GLI Procedure ME-12 and palladium analysis is performed using GLI Procedure ME-70 (inductively coupled plasma optical emission spectrometry using an Optima 5300 ICP OES analyzer or equivalent) with results reported in ppm (Ref: Galbraith Inc., 2323 Sycamore Drive, Knoxville, Tenn. 37921).

The names for the compounds of the present invention are generated using Symyx Version 3.2.NET with the IUPAC naming functionality.

Abbreviations represent the common and ordinary usage known to one of skill in the art and particular abbreviations used herein have the following meanings:
Boc or BOC tert-Butylcarbonyl
(Boc)₂O di-tert-Butylcarbonate
bs Broad singlet
d doublet
DAD diode array detector
dd doublet of doublets
DMAP dimethylaminopyridine
DMSO-d₆ hexadeuterodimethyl sulfoxide
HPLC high performance liquid chromatography
HRMS high resolution mass spectrometry
LCMS liquid chromatography mass spectrometry
NMR nuclear magnetic resonance
ppm parts per million
QT of quaternary time of flight
s singlet
t triplet
THF tetrahydrofuran
UPLC ultra-high performance liquid chromatography
GE General Electric
Ki Inhibition constant
PET positron emission tomography
T_{1/2} Half life
% ID/g Percent of injected dose per gram of tissue
USP United States Pharmacopeia
v/v volume to volume ratio
WFI Water for Injection.

### Preparation 1

### Synthesis of tert-butyl 7-bromo-5H-pyrido[4,3-b]indole-5-carboxylate, (IV)

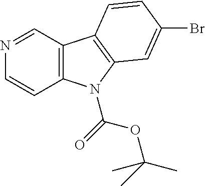 [SMILES: CC(C)(C)OC(=O)n1c2ccncc2c2ccc(Br)cc21]

A round bottomed flask is charged with 7-bromo-5H-pyrido[4,3-b]indole (15.00 g, 60.7 mmol), di-tert-butyl dicarbonate (19.87 g, 91.1 mmol, 1.5 eq), and dimethylaminopyridine (0.204 g, 1.8 mmol, 0.03 eq). Tetrahydrofuran (550 mL) is added and the resulting brown solution is allowed to stir at room temperature. The reaction is determined to be complete by LCMS after 4 hours. The reaction is concentrated to a brown solid. The solids are triturated in approximately 500 mL hexanes (with stirring), isolated by filtration, washed with hexanes, and then dried under high vacuum to afford the title compound as a dark tan solid (18.03 g, 86%). The material is used in the subsequent step without further purification. ¹H NMR (400 MHz, DMSO-d₆): δ 9.37 (d, J=1.2 Hz, 1H), 8.59 (d, Jₒ=6.0 Hz, 1H), 8.36 (d, J=1.2 Hz, 1H), 8.19 (dd, J=8.4 Hz, J=0.4 Hz, 1H), 7.99 (dd, Jₒ=6.0 Hz, Jₘ=1.2 Hz, 1H), 7.59 (dd, Jₒ=8.4 Hz, Jₘ=1.6 Hz, 1H), 1.66 (s, 9H). ¹³C NMR (100.6 MHz, CDCl₃): δ 150.06, 147.46, 143.37, 142.47, 139.07, 127.07, 122.51, 122.71, 121.27, 120.92, 119.67, 110.97, 85.67, 28.24. HRMS: Calc for C₁₆H₁₅N₂O₂Br (M+H)⁺ 347.0395, found 347.0400, Err=1.4 ppm.

### Preparation 2

### Synthesis of 3-(5-(tert-butoxycarbonyl)-5H-pyrido[4,3-b]indol-7-yl)pyridine 1-oxide, (III)

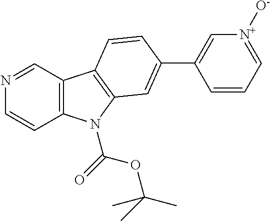 [SMILES: CC(C)(C)OC(=O)n1c2ccncc2c2ccc(-c3ccc[n+]([O-])c3)cc21]

A suspension of tert-butyl 7-bromo-5H-pyrido[4,3-b]indole-5-carboxylate (IV) (20.41 g, 58.8 mmol, 1.0 eq), bis(pinacolato)diboron (22.53 g, 88.7 mmol, 1.5 eq), and potassium acetate (19.55 g. 199.2 mmol, 3.4 eq) in dioxane (590 mL) is sparged with nitrogen for 15 minutes and treated with dichloro 1,1′-bis(diphenylphosphino)ferrocene palladium (II) dichloromethane adduct (4.82 g, 5.91 mmol, 0.1 eq). The mixture is sparged with nitrogen for an additional 10 minutes and the reaction is stirred at 80° C. overnight. After 17.5 hours, LCMS shows the reaction to be complete with the pinacol ester (tert-butyl 7-(4,4,5,5-tetramethyl-1,3,2-dioxaborolan-2-yl)-5H-pyrido[4,3-b]indole-5-carboxylate) representing 90% of the mixture (by UV). The reaction is cooled to room temperature. The mixture is sparged with nitrogen for 15 minutes and 3-bromo-pyridine 1-oxide (15.76 g, 90.6 mmol, 1.5 eq), sodium carbonate (156 mL of 2M in distilled water, 312 mmol, 5.3 eq), and palladium(II) tetrakistriphenylphosphine (3.40 g, 2.9 mmol, 5 mol %) are added. The reaction is stirred at 90° C. for 7.5 hours, cooled to room temperature and stirred overnight. The mixture is concentrated to a dark brown/black residue which is slurried and sonicated in a 90:10 methylene chloride:methanol solution (500 mL) and then filtered. The salts are washed with alternating portions of methylene chloride (2×250 mL) and methanol (2×250 mL), concentrated, and preabsorbed onto approximately 65 g silica gel. The material is divided and purified on two 330 g silica gel flash columns using a gradient of 100:0 (2 minute hold) to 95:5 methylene chloride:methanol over 10 minutes (8 minute hold) then increasing directly to 90:10 methylene chloride:methanol (25 minute hold). The material is concentrated and dried under high vacuum to afford a grey-black solid (9.01 g, 31%). This material is dissolved in 90:10 methylene chloride:methanol (400 mL) and stirred with Quadrasil-MP resin (26.05 g, 26.05-39.08 mmol thiol, 1-1.5 eq) overnight. The mixture is filtered and the resin washed with 90:10 methylene chloride:methanol (4×200 mL). The filtrates are concentrated, redissolved in 90:10 methylene chloride:methanol (400 mL) and stirred with fresh resin (6.48 g, 6.48-9.72 mmol thiol, 0.26-0.39 eq) overnight. The mixture is filtered and the resin washed with 90:10 methylene chloride:methanol (3×125 mL). The filtrates are concentrated and dried to afford a beige solid. The solids are dissolved in warm ethanol (800 mL, approximately 75° C.) and then allowed to cool to room temperature overnight. The resulting mixture is further cooled to 4° C. After 7 hours, the solids are isolated by filtration, washed with cold ethanol, and dried under high vacuum to afford the title compound as a beige solid (6.29 g). A second crop of crystals is isolated in the same manner (0.714 g) for a total of 7.00 g (78% recovery).

Palladium is further removed from combined batches of 3-(5-(tert-butoxycarbonyl)-5H-pyrido[4,3-b]indol-7-yl)pyridine 1-oxide. Multiple batches of 3-(5-(tert-butoxycarbonyl)-5H-pyrido[4,3-b]indol-7-yl)pyridine 1-oxide are combined (21.57 g, 59.7 mmol) and dissolved in 90:10 methylene chloride:methanol (990 mL). The golden brown solution is treated with Quadrasil-MP resin (56.0 g, 56.0-84.0 mmol thiol, 0.9-1.4 eq) overnight. The mixture is filtered and the resin washed with 90:10 methylene chloride:methanol (3×200 mL). The filtrates are concentrated and dried to afford the title compound as a cream colored solid (20.82 g, 97% recovery). ¹H NMR (400 MHz, CDCl₃): δ 9.31 (d, J=0.8 Hz, 1H), 8.67 (d, J=5.6 Hz, 1H), 8.64 (d, J=1.2 Hz, 1H), 8.59 (td, Jₘ=1.6 Hz, Jₚ=0.4 Hz, 1H), 8.23 (ddd, Jₒ=6.4 Hz, Jₘ=1.6 Hz, Jₘ=1.2 Hz, 1H), 8.17 (dd, Jₒ=8.0 Hz, Jₚ=0.4 Hz, 1H), 8.10 (dd, Jₒ=5.6 Hz, Jₘ=1.2 Hz, 1H), 7.60 (dd, Jₒ=8.0 Hz, Jₘ=1.6 Hz, 1H), 7.58 (ddd, Jₒ=6.4 Hz, Jₘ=1.6 Hz, Jₚ=0.8 Hz, 1H), 7.38 (ddd, Jₒ=8.0 Hz, Jₘ=6.4 Hz, Jₚ=0.4 Hz, 1H), 1.79 (s, 9H). ¹³C NMR (100.6 MHz, CDCl₃): δ 150.23, 147.74, 143.83, 142.90, 140.60, 139.13, 137.94, 137.77, 135.00, 125.88, 124.61, 124.47, 122.66, 121.25, 120.82, 115.11, 111.03, 85.68, 28.31. HRMS: Calc for C₂₁H₁₉N₃O₃ (M+H)⁺ 362.1505, found 362.1515, Err=2.8 ppm.

### EXAMPLE 1

### Synthesis of 5-(5-(tert-butoxycarbonyl)-5H-pyrido[4,3-b]indol-7-yl)-N,N,N-trimethylpyridin-2-aminium 4-methylbenzenesulfonate, (Ia)

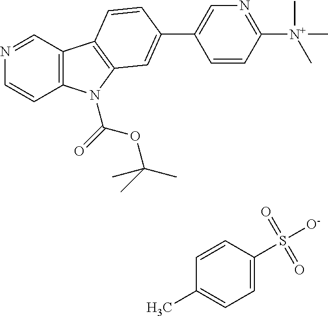

To a stirred solution of 3-(5-(tert-butoxycarbonyl)-5H-pyrido[4,3-b]indol-7-yl)pyridine 1-oxide (6.10 g, 16.9 mmol, 1.0 eq) in methylene chloride (435 mL) at room temperature is added p-toluenesulfonic anhydride (12.50 g, 38.3 mmol, 2.0 eq) in a single portion. The orange brown reaction mixture becomes homogeneous and is stirred for 30 minutes. A 1.0 M solution of trimethylamine in tetrahydrofuran (335 mL, 335 mmol, 20.0 eq) is added slowly (note—slight exotherm). The resulting solution is stirred for 30 minutes and then treated with additional p-toluenesulfonic anhydride (5.49 g, 16.9 mmol, 1.0 eq) in a single portion. After 30 minutes, a third portion of p-toluenesulfonic anhydride (5.49 g, 16.9 mmol, 1.0 eq) is added. After an additional 30 minutes, a final portion of p-toluenesulfonic anhydride (5.49 g, 16.9 mmol, 1.0 eq) is added and the mixture/solution is stirred for 30 minutes. LCMS monitoring shows complete consumption of the starting material at this point (m/z=306, M-tBu). The reaction mixture is concentrated to a light yellow solid under reduced pressure. The solid is slurried in methylene chloride (500 mL) and extracted two times with water (300 mL, 200 mL). The aqueous layers are combined and extracted with methylene chloride (2×200 mL). The combined organic phases are dried over sodium sulfate, filtered, and concentrated to a tan/orange solid. The solids are dissolved in methylene choride (75 mL) and added dropwise to rapidly stifling diethyl ether (600 mL). The resulting solids are isolated by filtration and washed with diethyl ether. The trituration process is repeated 1 to 3 more times as necessary to remove any additional tosyl-related impurities. When required, the material is further purified on a 40 g silica gel column (2-4 g compound) using a gradient of 100:0 (3 minute hold) to 90:10 methylene chloride:methanol over 3 minutes with a 3 minute hold followed by increasing to 80:20 methylene chloride:methanol over 3 minutes with a 20 minute hold. Fractions containing pure product are combined, concentrated, redissolved in methylene chloride, filtered to remove residual silica gel, and then concentrated. After drying under high vacuum, the compound Ia is obtained as a beige solid (6.73 g, 69%).

### Purification of 5-(5-(tert-butoxycarbonyl)-5H-pyrido[4,3-b]indol-7-yl)-N,N,N-trimethylpyridin-2-aminium 4-methylbenzenesulfonate

Multiple batches of 5-(5-(tert-butoxycarbonyl)-5H-pyrido[4,3-b]indol-7-yl)-N,N,N-trimethylpyridin-2-aminium 4-methylbenzenesulfonate are combined (11.25 g) and dissolved in methylene chloride (500 mL). The turbid orange mixture is dried over Na₂SO₄ and filtered through Celite®. The filter cake is washed with methylene chloride (100 mL). The resulting orange solution is added dropwise to rapidly stirring diethyl ether (2 L). The precipitated solids are isolated by filtration and washed with diethyl ether (1 L). After drying under high vacuum, the title compound is obtained as a beige solid (10.75 g, 96% recovery). ¹H NMR (400 MHz, CDCl₃): δ 9.31 (dd, J<0.5 Hz (2), 1H), 8.77 (dd, J=2.4, 0.6 Hz, 1H), 8.67 (dd, J=5.9, <0.5 Hz, 1H), 8.63 (dd, J=1.6, 0.5 Hz, 1H), 8.51 (dd, J=8.7, 0.6 Hz, 1H), 8.16 (dd, J=8.1, 0.5 Hz, 1H), 8.13 (dd, J=8.7, 2.4 Hz, 1H), 8.11 (dd, J=5.9, <0.5 Hz, 1H), 7.80 (para d, J=8.1 Hz, 2H), 7.58 (dd, J=8.1, 1.6 Hz, 1H), 7.13 (para d, J=8.1, 2H), 3.94 (s, 9H), 2.28 (s, 3H), 1.78 (s, 9H). ¹³C NMR (100.6 MHz, CDCl₃): δ 156.0, 150.2, 147.6, 146.6, 144.0, 143.9, 142.8, 139.6, 139.3 (2), 139.2, 135.2, 128.7, 126.0, 124.3, 123.1, 121.3, 120.9, 116.0, 115.3, 111.1, 85.7, 55.4, 28.3, 21.2. HRMS: Calc for C₂₄H₂₇N₄O₂ (parent ion, M+H)⁺ 403.2134, found 403.2129, Err=−1.2 ppm. Elemental Analysis (GLI Procedure ME-12): Calc C 64.79 H 5.96 N 9.75, Found C 63.88/63.44 H 6.05/5.90 N 9.34/9.24. Pd Analysis (GLI Procedure ME-70): 4.6 ppm (Galbraith Inc., 2323 Sycamore Drive, Knoxville, Tenn. 37921).

### EXAMPLE 2

### Radiosynthesis of 7-(6-[¹⁸F]fluoro-3-pyridyl)-5H-pyrido[4,3-b]indole, [¹⁸F]T807

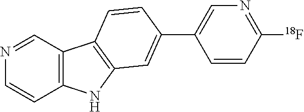 [SMILES: [18F]c1ccc(-c2ccc3c(c2)[nH]c2ccncc23)cn1]

The title compound is prepared using an automated radiosynthesizer such as a GE TRACERIab FX_{F-N} automated radiosynthesizer. Typical decay corrected yield is 45-55% [¹⁸F]Fluoride activity is retained on a Sep-Pak® Light Accell™ Plus (QMA) Carbonate Cartridge (46 mg Sorbent per Cartridge, 40 μm Particle Size) and eluted to the reaction vessel using 0.8 mL of an aqueous Cryptand 2.2.2-K₂CO₃ solution [Cryptand 2.2.2 (7 mg) and potassium carbonate (0.75 mg) in WFI (water for injection, 0.4 mL) and acetonitrile (0.4 mL)]. The eluted activity is dried by heating at 70° C. under nitrogen flow and vacuum for 4.5 minutes. The temperature is then raised to 100° C. and kept for an additional minute. Nitrogen flow is turned off and the activity is dried under vacuum for 4 minutes to afford anhydrous Cryptand 2.2.2-K₂CO₃ [¹⁸F]fluoride.

A solution of 5-(5-(tert-butoxycarbonyl)-5H-pyrido[4,3-b]indol-7-yl)-N,N,N-trimethylpyridin-2-aminium 4-methylbenzenesulfonate [1.5 mg in anhydrous DMSO (2 mL)] is added to the reaction vessel and the resulting mixture is kept at 110° C. for 5 minutes followed by de-protection using 1 mL of 3 N HCl_{(aq)} at 100° C. for 5 minutes. After cooling to 50° C., the crude title compound is neutralized with 7 mL of 0.5 M NaOH_{(aq)} (3.5 mL of 1 N NaOH_{(aq)} and with 3.5 mL of water for injection, WFI). The resulting mixture is passed through an Oasis® HLB Light reversed-phase cartridge (30 mg Sorbent per Cartridge, 30 μm Particle Size). The retained crude title compound is washed with WFI (5 mL) then eluted off the Oasis HLB Light cartridge using acetonitrile (1.5 mL). The crude material is diluted with WFI (3 mL) then loaded onto a semi-preparative C-18 reversed-phase HPLC column (Agilent ZORBAX Eclipse XDB-C18 9.4×250 mm, flow rate=4 mL/min) for purification (RT˜8 minutes) using the isocratic elution of 40% acetonitrile in 10 mM ammonium acetate/water.

The HPLC fraction containing the title compound is collected and diluted with 30 mL of WFI. The diluted solution is then passed through a Sep-Pak Light C18 reversed-phase Cartridge (130 mg Sorbent per Cartridge, 55-105 μm Particle Size) and the retained title compound is washed with 5 mL of WFI. ¹⁸F-AV-1451 is eluted off the Sep-Pak C18 Plus Light cartridge using dehydrated alcohol, USP (1 mL) followed by 0.9% Sodium Chloride Injection, USP (2 mL) into 0.9% Sodium Chloride Injection, USP (7 mL). The drug substance solution is transferred into the bulk product vial (BPV) through a 0.22 um sterilizing filter (Millex GV PVDF, Millipore SLGV013SL).

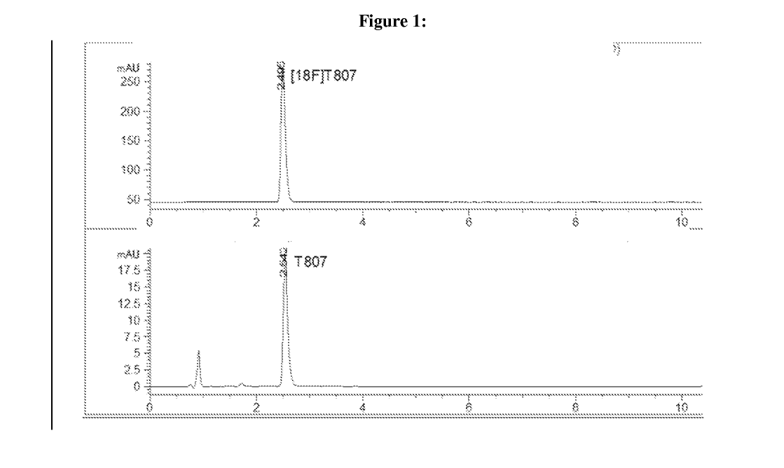

The HPLC method of analysis utilizes isocratic elution with a 25%:75% v/v acetonitrile:water with 0.1% TFA mobile phase on a C18 reversed-phase HPLC column with a flow rate of 1.0 mL/min. Two detectors, a radiometric detector and a UV detector set at 270 nm, are fitted to the system. Injections of Reference Standard solutions and Drug Product sample are made. The peaks from the UV chromatograms and radiochromatogram are integrated, and the data is used to calculate the radiochemical purity and specific activity and confirm the radiochemical identity. The identity of the title compound is confirmed by matching the HPLC UV and radio-chromatographic retention times obtained with ¹⁸F-labeled compound and non-radioactive 7-(6-fluoro-3-pyridyl)-5H-pyrido[4,3-b]indole reference standard. See FIG. 1.

### EXAMPLE 3

### Film Autoradiography of AD Brain Tissue Sections

Film autoradiography of AD Brain Tissue Sections are performed in a manner consistent with previously published methods (See Zhang, W., et al. F-18 stilbenes as PET imaging agents for detecting beta-amyloid plaques in the brain. Journal of Medicinal Chemistry, 48: 5980-5988, 2005, Zhang, W., et al. F-18 stilbenes as PET imaging agents for amyloid plaque imaging. Nucl Med Biol. 2007 34(1):89-97.)

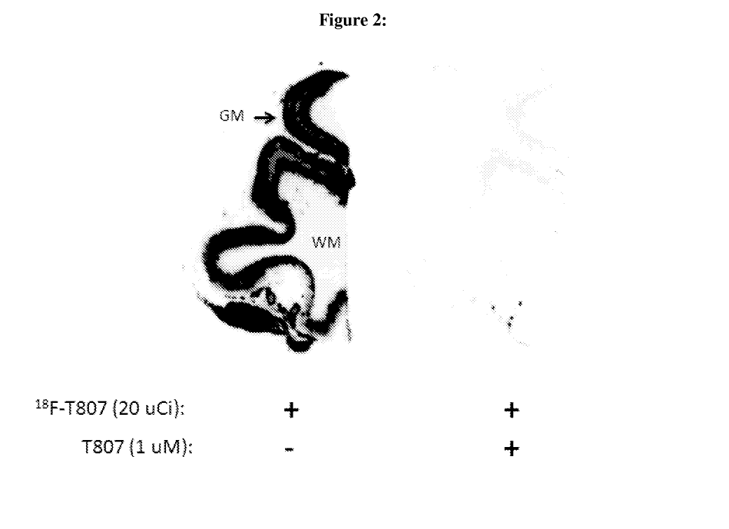

Postmortem AD diagnosed human brain sections (frontal lobe, 10 um) are covered with 0.5 ml of [¹⁸F]T807 (in 2.5:2.5:95=DMSO:ethanol:1X-phospate buffered saline (PBS), about 20 uCi of [¹⁸F]T807 per slide, and incubated for 60 minutes at room temperature. Then successive washing cycles are employed with 2 minutes of 1× PBS, 2 minutes of 30% ethanol/1× PBS, 2 minutes of 70% ethanol/1× PBS, and 2 minutes 1× PBS, to remove any unbound tracer. After drying under a fume hood, the sections are put on a FujiFilm radio-sensor cassette and exposed over night. The autoradiography signal is recorded in the radio-sensor sheet and is read/visualized with the FujiFilm Bio-Imaging System FLA-7000. Autoradiographic visualization of tau is observed in gray matter of postmortem AD brain tissue. See FIG. 2 which illustrates [¹⁸F]T807 labeling of tau in post-mortem frontal lobe sections (10 um) of tau-positive human brain tissue from patients with AD, approximately 20 uCi of [¹⁸F]T807 per slide. Strong autoradiography signal of [¹⁸F]T807 is observed on the grey matter (GM) region, and the presence of tau in these regions are confirmed by tau-immunostaining. The non-specific or background [¹⁸F]T807 signal is shown in the white matter region (WM) and is low. The specificity of the autoradiography signal, with respect to binding to native tau aggregates, is indicated by the blocking effect of 1 uM of cold T807.

We claim:

1. A compound of the formula: 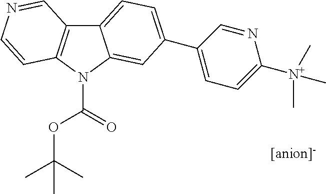 [SMILES: CC(C)(C)OC(=O)n1c2ccncc2c2ccc(-c3ccc([N+](C)(C)C)nc3)cc21]
wherein [anion]⁻ is a suitable anionic counterion.

2. A compound of claim 1 of the formula: 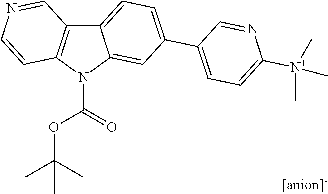 [SMILES: CC(C)(C)OC(=O)n1c2ccncc2c2ccc(-c3ccc([N+](C)(C)C)nc3)cc21]
wherein [anion]⁻ is an alkyl sulfonate or aryl sulfonate.

3. A compound of claim 2 which is 5-(5-(tert-butoxycarbonyl)-5H-pyrido[4,3-b]indol-7-yl)-N,N,N-trimethylpyridin-2-aminium 4-methylbenzenesulfonate, represent by the formula:

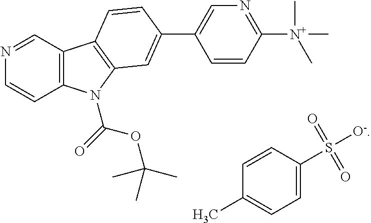

4. A compound of claim 2 which is 5-(5-(tert-butoxycarbonyl)-5H-pyrido[4,3-b]indol-7-yl)-N,N,N-trimethylpyridin-2-aminium 4-methylsulfonate, represent by the formula:

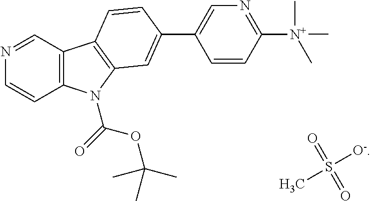

5. A process of making a compound of the formula: 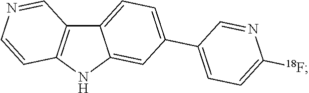
comprising reacting a compound of the formula: 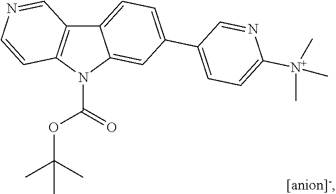 [SMILES: CC(C)(C)OC(=O)n1c2ccncc2c2ccc(-c3ccc([N+](C)(C)C)nc3)cc21]
wherein [anion]⁻ is a suitable anionic counterion, with [¹⁸F]fluoride.

6. A process of making a compound of the formula: 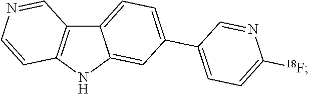
comprising reacting 5-(5-(tert-butoxycarbonyl)-5H-pyrido[4,3-b]indol-7-yl)-N,N,N-trimethylpyridin-2-aminium 4-methylbenzenesulfonate, represented by the formula: 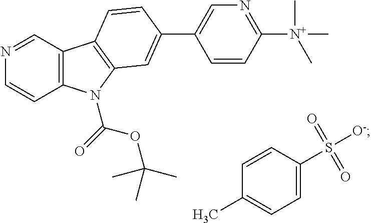
with [¹⁸F]fluoride.

7. A process of making a compound of the formula: 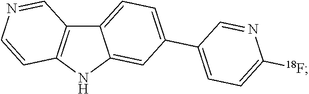
comprising reacting 5-(5-(tert-butoxycarbonyl)-5H-pyrido[4,3-b]indol-7-yl)-N,N,N-trimethylpyridin-2-aminium 4-methylsulfonate, represented by the formula: 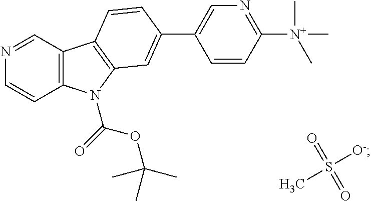
with [¹⁸F]fluoride.

8. A diagnostic composition comprising 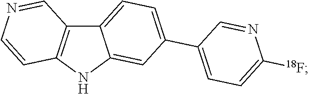
made by the process of claim 5, 6, or 7,
and a pharmaceutically acceptable carrier or diluent.

9. A diagnostic composition comprising 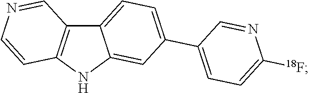
made by the process of claims 5, 6, or 7;
and 10% (v/v) ethanol, 90% (w/v) (0.9% aqueous Sodium Chloride).

10. A diagnostic composition comprising 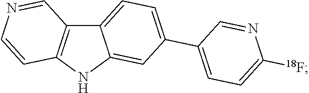
made by the process of claims 5, 6, or 7;
and 10% (v/v) ethanol/90% (21 mM sodium phosphate).

11. A diagnostic composition comprising 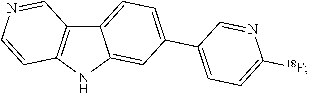
made by the process of claims 5, 6, or 7;
and 9% (v/v) ethanol, 1% (w/v) Kolliphor HS 15, and 90% (v/v) (0.9% aqueous Sodium Chloride).

12. A method of imaging tau comprising:
a. introducing into a mammal a detectable quantity of the compound: 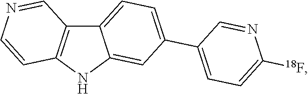
made by the process of claim 5, 6, or 7;
b. allowing sufficient time for said compound to become associated with tau; and
c. detecting said compound.

13. A method of imaging tau comprising:
d. introducing into a mammal a detectable quantity of a diagnostic composition of claim 8, 9, 10 or 11;
e. allowing sufficient time for said diagnostic composition to become associated with tau; and
f. detecting the diagnostic composition.

14. An intermediate for preparing a compound of claim 1 wherein the intermediate is 3-(5-(tert-butoxycarbonyl)-5H-pyrido[4,3-b]indol-7-yl)pyridine 1-oxide, represented by the formula: [chemical structure]
